(19) United States
(12) Patent Application Publication
(10) Pub. No.: US 2025/0367421 A1
(43) Pub. Date: Dec. 4, 2025

(54) SODIUM HYALURONATE-CONTAINING OPHTHALMIC MICRONEEDLE PATCH, AND PREPARATION METHOD AND USE THEREOF

(71) Applicant: Wenzhou Medical University, Wenzhou City (CN)

(72) Inventors: Kaihui NAN, Wenzhou City (CN); Sen Lin, Wenzhou City (CN); Baoshan HUANG, Wenzhou City (CN)

(73) Assignee: Wenzhou Medical University, Wenzhou City (CN)

(21) Appl. No.: 19/083,109

(22) Filed: Mar. 18, 2025

(30) Foreign Application Priority Data
May 29, 2024 (CN) 202410677871.0

(51) Int. Cl. A61M 37/00; A61K 47/32; A61K 47/36

(52) U.S. Cl. CPC A61M 37/0015; A61K 47/32; A61K 47/36; A61M 2037/0053; A61M 2037/0061

(57) ABSTRACT

Provided is a sodium hyaluronate-containing ophthalmic microneedle patch, and a preparation method and use thereof. The preparation method includes: preparing a microneedle patch matrix solution, where the microneedle patch matrix solution includes sodium hyaluronate and polyvinyl alcohol (PVA); and dropping the microneedle patch matrix solution into a microneedle patch mold, drying, and then demolding to obtain the sodium hyaluronate-containing ophthalmic microneedle patch.

### CROSS REFERENCE TO RELATED APPLICATION

This patent application claims the benefit and priority of Chinese Patent Application No. 202410677871.0 filed with the China National Intellectual Property Administration on May 29, 2024, the disclosure of which is incorporated by reference herein in its entirety as part of the present application.

### TECHNICAL FIELD

The present disclosure relates to the field of pharmaceutical technologies, and in particular to a sodium hyaluronate-containing ophthalmic microneedle patch, and a preparation method and use thereof.

### BACKGROUND

Sodium hyaluronate eye drops are one of the most widely-used artificial tears in clinical practice. Sodium hyaluronate is an acidic mucopolysaccharide widely distributed in the extracellular matrix of animal and human connective tissues. More importantly, sodium hyaluronate is also an important component of the corneal stroma, and plays an important role in the growth and development of corneal cells, the inflammation inhibition, the tissue repair and healing, etc. Because carboxyl and hydroxyl groups in a molecular structure of sodium hyaluronate can form hydrogen bonds with water, sodium hyaluronate exhibits excellent water-retention properties. As a result, sodium hyaluronate is widely used to improve the moisturizing ability of corneas and the irritative symptoms of eyes. In addition, sodium hyaluronate eye drops are a polymer polysaccharide biomaterial produced through the repeated arrangement of N-acetylglucuronic acid units. Sodium hyaluronate eye drops can bind to fibronectin to enhance the adhesion and extensibility of corneal epithelial cells and improve the self-repair ability of corneas.

Commercial sodium hyaluronate eye drops are widely used in clinical practice with a concentration of 0.1%, and can effectively improve the mild corneal wound such as mild dry eye disease. However, these commercial sodium hyaluronate eye drops have a poor therapeutic effect for severe corneal wound induced by factors such as long-term application of preservative-containing eye drop preparations, after glaucoma surgery, and after cataract surgery. The above phenomenon is attributed to short ocular surface retention time of eye drop preparations, poor corneal barrier penetration, poor patient compliance caused by frequent dropping, etc., which make it difficult to effectively improve the sodium hyaluronate contents in corneal epithelial layers and stromata. As a result, the corneal epithelial layers and stromata continue to be in a highly-inflammatory environment, which further leads to keratitis, corneal ulcer, corneal scarring, etc., and eventually causes the complete blindness. Therefore, there is an urgent need to develop a new dosage form of sodium hyaluronate for eyes with high safety and high corneal permeability.

Microneedles are a minimally-invasive and painless drug delivery manner with a length of 25 μm to 2,000 μm, which can effectively break the physical barrier of a tissue and efficiently deliver a drug to a target site. When applied to the anterior segments of eyes, microneedles can penetrate the tear film and cornea, which can directly increase the penetration of a drug. A microneedle patch is produced by connecting a plurality of microneedles with a substrate in an array manner. A microneedle patch can make a sufficient amount of drugs penetrate and directly release to a target site, which maximizes the exertion of functions of the drug itself. Ophthalmic microneedle patches are a painless and minimally-invasive drug delivery manner and are disposable. Ophthalmic microneedle patches can effectively overcome the dilemma that the too-frequent dropping of eye drops would result in poor patient compliance. Therefore, the use of sodium hyaluronate in ophthalmic microneedle patches to improve the corneal permeability shows a promising application prospect.

However, sodium hyaluronate is a tough material. When used to prepare microneedle patches, sodium hyaluronate allows the advantages of flexibility and strong plasticity, but also leads to defects such as low needle formation rate and insufficient hardness. In order to solve the above problem, the inventors attempt to introduce polyvinyl alcohol (PVA) into a microneedle patch matrix solution to enhance the hardness of soluble microneedles.

### SUMMARY

In order to overcome the problem that the sodium hyaluronate eye drops with a concentration of 0.1% in the prior art cannot allow the preparation of a microneedle patch with a complete morphology and a specified mechanical strength due to the toughness characteristic and low concentration limitation of sodium hyaluronate itself, the present disclosure provides a sodium hyaluronate-containing ophthalmic microneedle patch, and a preparation method and use thereof. The present disclosure provides the following technical solutions:

In a first aspect, the present disclosure provides a method for preparing a sodium hyaluronate-containing ophthalmic microneedle patch, including the following steps:
preparing a microneedle patch matrix solution, where the microneedle patch matrix solution includes sodium hyaluronate and PVA; and
dropping the microneedle patch matrix solution into a microneedle patch mold, drying, and then demolding to obtain the sodium hyaluronate-containing ophthalmic microneedle patch.

In some embodiments, a total matrix solid content in the microneedle patch matrix solution is in a range of 5% to 15%.

In some embodiments, the total matrix solid content in the microneedle patch matrix solution is 10%.

In some embodiments, the PVA in the microneedle patch matrix solution accounts for 40% to 99% of the total matrix solid content (which is a total solid content of dissolved sodium hyaluronate and PVA in a matrix solution).

In a second aspect, the present disclosure provides a sodium hyaluronate-containing ophthalmic microneedle patch prepared by the method as described above.

In a third aspect, the present disclosure provides use of the sodium hyaluronate-containing ophthalmic microneedle patch as described above in a drug for treating a severe corneal wound.

In some embodiments, the severe corneal wound includes at least one selected from the group consisting of the severe corneal wound caused by long-term preservative application, severe dry eye disease, the severe corneal wound caused by alkali burn, the severe corneal wound caused by a mechanical trauma, bacterial keratitis, and fungal keratitis.

In summary, in the present disclosure, a microneedle patch matrix solution is prepared by mixing PVA with sodium hyaluronate, and then poured into a mold to obtain a sodium hyaluronate-containing ophthalmic microneedle patch. The PVA is a water-soluble polymer produced from the hydrolysis of polyvinyl acetate. The PVA shows excellent biocompatibility and biodegradability, and can effectively improve the needle formation rate and mechanical properties of the microneedle patch. Soluble microneedles prepared with the microneedle patch matrix solution of these two components not only show high hardness, but also have the toughness characteristic of sodium hyaluronate, which makes the microneedle patch have flexibility and plasticity. Thus, the microneedle patch shows excellent practicability for the treatment of uneven or curved tissues. The present disclosure effectively overcomes the technical limitation that the conventional commercial sodium hyaluronate eye drops with a concentration of 0.1% cannot be gelatinized to form a microneedle patch.

The present disclosure has the following beneficial effects: The sodium hyaluronate-containing ophthalmic microneedle patch shows excellent mechanical properties, can effectively break the corneal epithelial tissue barrier, and efficiently deliver sodium hyaluronate into the corneal tissue, which effectively improves the defect that the eye drop dosage form has low bioavailability at an ocular tissue.

Test results show that the sodium hyaluronate-containing ophthalmic microneedle patch can effectively treat the severe corneal wound in rats, effectively inhibit the infiltration of inflammatory cells in the corneal stroma, and overcome the limitation that sodium hyaluronate eye drops are difficult to repair the deep corneal wound. Therefore, the sodium hyaluronate-containing ophthalmic microneedle patch shows an important application value and a promising application prospect.

### BRIEF DESCRIPTION OF THE DRAWINGS

In order to more clearly illustrate the technical solutions in the embodiments of the present disclosure or in the prior art, the drawings required for description in the embodiments or the prior art will be briefly described below. It is apparent that the drawings in the following description are only some embodiments of the present disclosure. Other drawings obtained according to these drawings by those skilled in the art without any inventive work still belong to the scope of the present disclosure.

FIGS. 1A to 1C show a microneedle patch with 49 microneedles (7×7 array) that is designed by using 3D (Three-Dimensional) max, where FIG. 1A shows an overall view of the microneedle patch, FIG. 1B shows sizes of a microneedle body and a backing, and FIG. 1C shows edge lengths of the microneedle patch, an arrangement of the microneedle array, and a spacing between adjacent microneedles;

FIG. 2 shows that PVA-free 0.1% commercial sodium hyaluronate eye drops are transformed from a liquid state to a viscous state in a microneedle patch female mold (PDMS female mold), and cannot be demolded to form a microneedle patch morphology;

FIG. 3 shows microneedle patches produced after demolding in Examples 1, 2, 3, and 4 sequentially from left to right;

FIG. 4 shows mechanical property test results of the three sodium hyaluronate-containing microneedle patches in Examples 2, 3, and 4;

FIG. 5 shows compression flexibility test results of the three sodium hyaluronate-containing microneedle patches in Examples 2, 3, and 4;

FIG. 6 shows a comparison diagram for a microneedle patch before and after penetration for rat corneas;

FIG. 7 shows a microscopic image of a stained corneal epithelial tissue penetrated by a microneedle patch;

FIGS. 8A to 8B show images of corneal tissues acquired by z-axis scanning under a confocal microscope after an eye drop dosage form is applied to and a microneedle patch dosage form penetrates through surfaces of eyeballs in rats, respectively;

FIGS. 9A to 9B show fluorescence intensity test results of rat eyes;

FIG. 10 shows corneal healing conditions in rats;

FIGS. 11A to 11E show corneal repair ability test results, where FIG. 11A shows an effect of a sodium hyaluronate-containing microneedle patch to effectively inhibit the corneal epithelial damage, FIG. 11B shows effects of the sodium hyaluronate-containing microneedle patch to effectively inhibit the corneal edema and inflammation, FIG. 11C shows statistical results of the sodium hyaluronate-containing microneedle patch to effectively inhibit the corneal epithelial damage, FIG. 11D shows statistical results of the sodium hyaluronate-containing microneedle patch to effectively inhibit the corneal edema, and FIG. 11E shows statistical results of the sodium hyaluronate-containing microneedle patch to effectively inhibit the corneal inflammation; and

FIG. 12 shows an effect of a sodium hyaluronate-containing microneedle patch to effectively reduce the infiltration of inflammatory cells in the corneal stroma.

### DETAILED DESCRIPTION OF THE EMBODIMENTS

In order to make the objects, technical solutions, and advantages of the present disclosure clear, the present disclosure will be further described in detail below with reference to the examples and accompanying drawings.

### Mold Preparation

### 1. Design of a Size of a Microneedle Patch and Preparation of a Male Mold

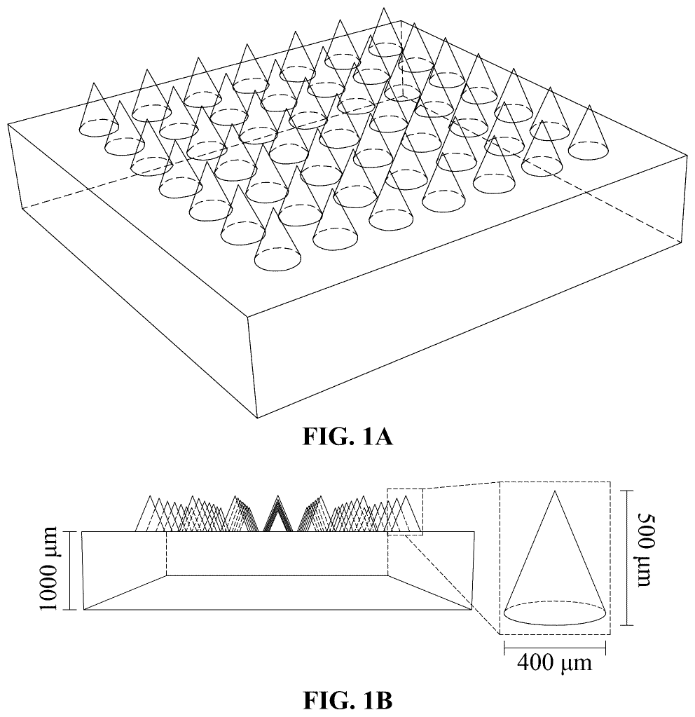

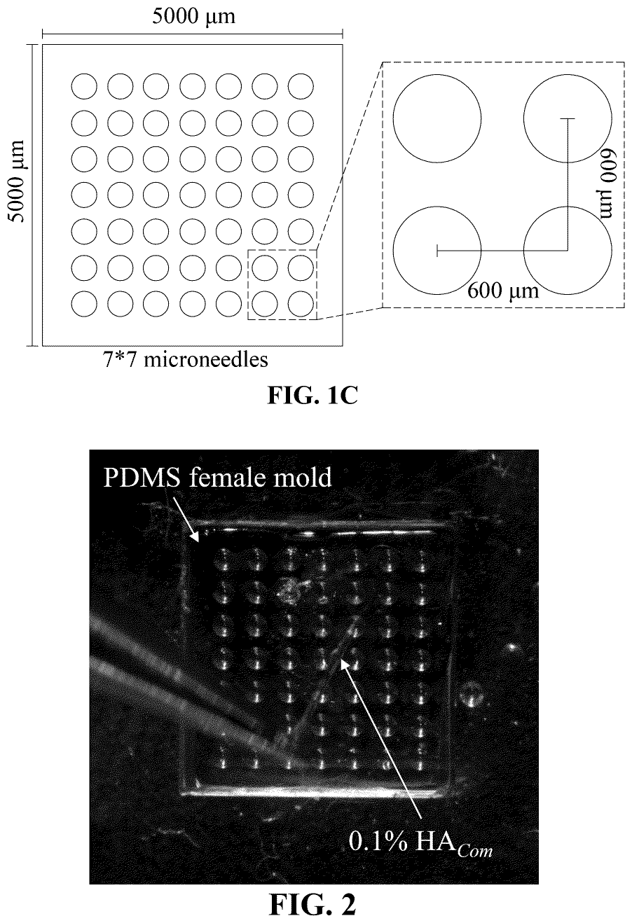

A microneedle patch with 49 microneedles (7×7 array) was designed with the 3D modeling software 3D max. As shown in FIGS. 1A to 1C, each of the microneedles was a cone with a height of 500 μm and a bottom radius of 200 μm, a spacing between two adjacent microneedles was 200 μm, and a length, a width, and a height of a backing were 5 mm×5 mm×1 mm. Through a 3D printing technology, a polymer resin material was prepared into a microneedle patch male mold with the above size.

### 2. Preparation of a Microneedle Patch Female Mold

Polydimethylsiloxane (PDMS) and a curing agent were fully mixed in a volume ratio of 10:1, a resulting mixture was centrifuged at 3,500 rpm for 5 min to remove air bubbles, and then injected into the male mold. Then the male mold was centrifuged at 3,500 rpm for 5 min to remove air bubbles, then heat-cured on a constant-temperature heating stage at 85° C. for 2 h, and cooled to room temperature. A resulting female mold was separated from the male mold to obtain the microneedle patch female mold.

### Comparative Examples and Examples

The inventors carried out a plurality of experiments to explore the performance differences among microneedle patches prepared with sodium hyaluronate and PVA at different amounts and concentration ratios. Four representative experiments were selected as examples to demonstrate the impacts of a total matrix solid content and a concentration ratio on the performance of a microneedle patch, which were specifically as follows:

### 1. Comparative Example 1 and Examples 1 to 2

Preparation of microneedle patch matrix solutions: (1) With a sodium hyaluronate concentration fixed to 0.1%, a PVA concentration was adjusted: 5 mL of commercial sodium hyaluronate eye drops with a concentration of 0.1% was taken, 0 mg (0%, Comparative Example 1), 245 mg (4.9%, Example 1), and 495 mg (9.9%, Example 2) of a PVA powder were added respectively, and thorough stirring was conducted.

### 2. Examples 3 to 4

Preparation of microneedle patch matrix solutions: (2) With a total matrix solid content fixed to 10%, a ratio of sodium hyaluronate to PVA was adjusted: 5 mL of each of sodium hyaluronate solutions with concentrations respectively of 3% and 6% was taken, 350 mg (7%, Example 3) and 200 mg (4%, Example 4) of a PVA powder were added respectively, and thorough stirring was conducted.

50 μL of each of the matrix solutions prepared in Comparative Example 1 and Examples 1 to 4 was poured into a groove of a PDMS female mold and centrifuged at 3,500 rpm for 10 min. Each of the matrix solutions was added in the same volume, and the PDMS female mold was centrifuged. This process was repeated 3 times. Resulting products each were dried in an oven at 37° C. for 12 h. Resulting microneedle patches each were separated from the PDMS female mold by tweezers under a stereo microscope, and photographed for morphology.

As shown in FIG. 2, the PVA-free 0.1% commercial sodium hyaluronate eye drops in Comparative Example 1 are transformed from a liquid state to a viscous state in the microneedle patch female mold, but cannot be demolded to form a microneedle patch morphology.

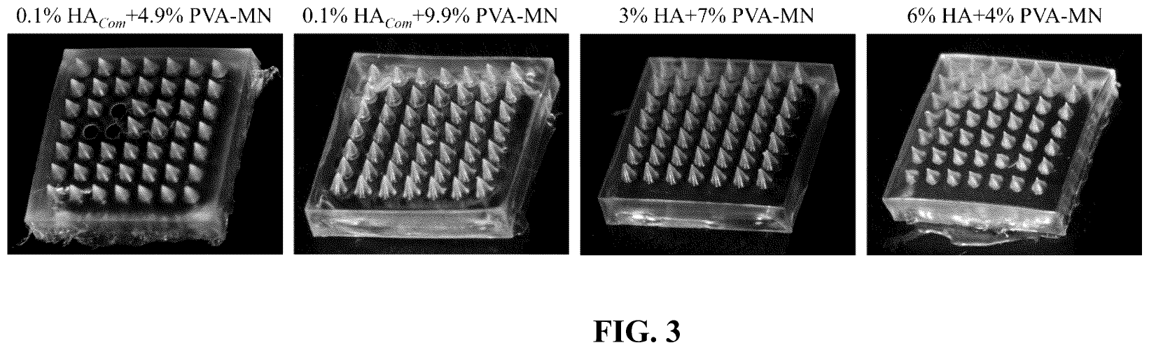

As shown in FIG. 3: In Example 1, 0.1% commercial sodium hyaluronate eye drops with 4.9% PVA can allow the successful demolding to produce a microneedle patch, where a part of needles are easy to fall off. In Example 2, 0.1% commercial sodium hyaluronate eye drops with 9.9% PVA can allow the successful demolding to produce the microneedle patch with a needle formation rate of 100%. In Examples 3 to 4, with the total matrix solid content fixed to 10%, a sodium hyaluronate concentration of 3% and a PVA concentration of 7%, and a sodium hyaluronate concentration of 6% and a PVA concentration of 4% both can allow the successful demolding to produce microneedle patches with a needle formation rate of 100%.

### Test Examples

### 1. Mechanical Properties and Flexibility of Sodium Hyaluronate-Containing Microneedle Patches

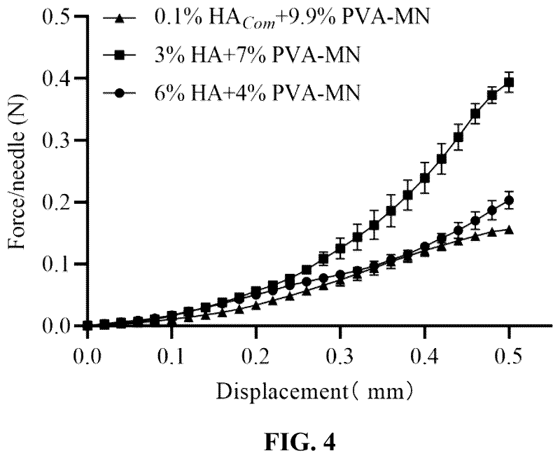

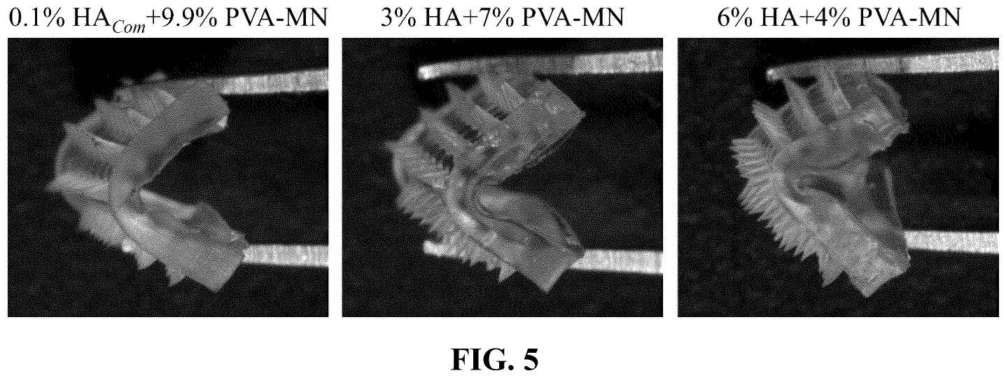

Sodium hyaluronate-containing microneedle patches each were placed flatly on a stainless-steel platform of a texture analyzer with needle tips facing upward. With a cylindrical probe of a diameter of 10 mm, an external force was applied at a constant speed of 100 μm/s in a direction perpendicular to a microneedle patch. A preset pressure application-terminating distance was 500 μm. As shown in FIG. 4, the three sodium hyaluronate-containing microneedle patches in Examples 2, 3, and 4 do not break during the entire pressure application process, and show strong mechanical properties. The microneedle patches each were subjected to a compression flexibility test with tweezers under a same force. As shown in FIG. 5, the three sodium hyaluronate-containing microneedle patches in Examples 2, 3, and 4 all show excellent flexibility. For the follow-up therapeutic effect evaluation, a 0.1% sodium hyaluronate-containing microneedle patch is taken as an example.

The sodium hyaluronate-containing microneedle patch prepared in Example 2 is selected below for animal experiments to verify a therapeutic effect of the sodium hyaluronate-containing microneedle patch:

### 2. Corneal Penetration Performance of the Sodium Hyaluronate-Containing Microneedle Patch

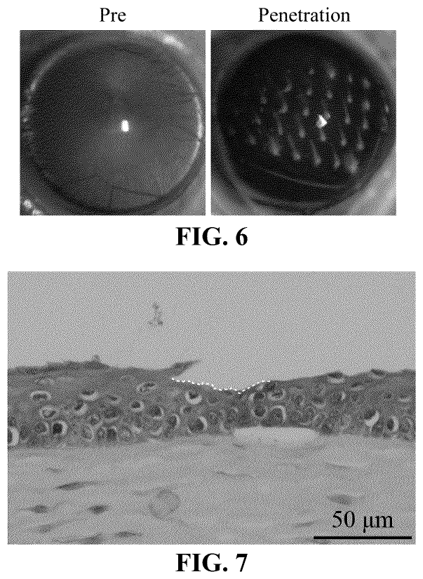

SD rats were anesthetized with a 1% sodium pentobarbital solution. The sodium hyaluronate-containing microneedle patch was applied to a surface of an eyeball of a rat, gently pressed with the thumb for 60 s, and then removed. In order to evaluate the corneal penetration performance of the sodium hyaluronate-containing microneedle patch, the sodium fluorescein spot staining method and the hematoxylin and eosin staining (H&E staining) method were used to evaluate a corneal defect degree. a. Sodium fluorescein spot staining: 1 μL of a sodium fluorescein solution with a concentration of 2 mg/mL was spotted into the conjunctival sac of a rat, and observed under a cobalt blue filter using a slit lamp microscope. As shown in FIG. 6, there is no residual sodium fluorescein before the microneedle patch penetrates (Pre group), indicating that the corneal epithelium is intact. After the microneedle patch penetrates (penetration group), there is a square dot-like array of dozens of fluorescein sodium-stained spots, indicating that the penetration of the microneedle patch successfully breaks the corneal epithelial barrier. b. H&E staining: Rats were euthanized. Then eyeballs were rapidly isolated, cleaned with normal saline, and treated with a fixation solution. A complete cornea was isolated under a stereo microscope, dehydrated, embedded, and sectioned to obtain paraffin-embedded section samples. The paraffin-embedded section samples were subjected to treatments such as dewaxing, dehydration, hematoxylin staining, acetic acid differentiation, blue returning, eosin staining, dehydration, and mounting in sequence, and then observed and photographed under a microscope. As shown in FIG. 7, after being penetrated by the microneedle patch, the corneal epithelial tissue undergoes an obvious defect, which further indicates that the penetration of the microneedle patch successfully breaks the corneal epithelial barrier.

3. Efficient Delivery of Sodium Hyaluronate by the Microneedle Patch Dosage Form into a Deep Corneal Layer

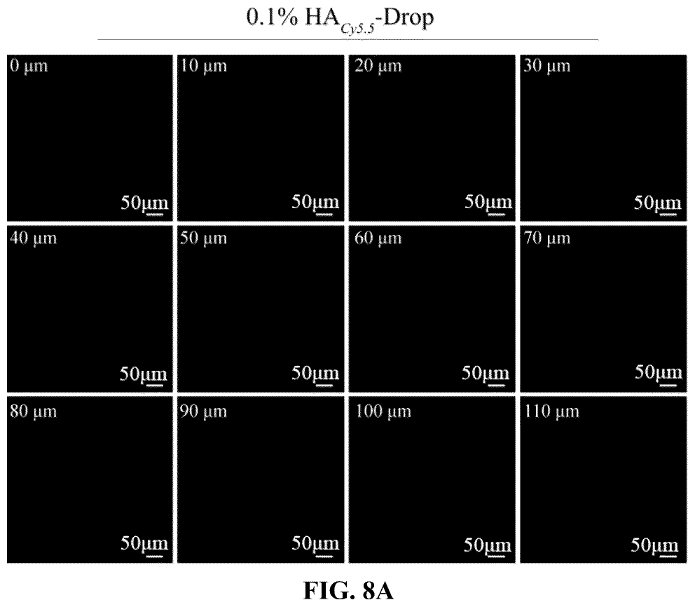

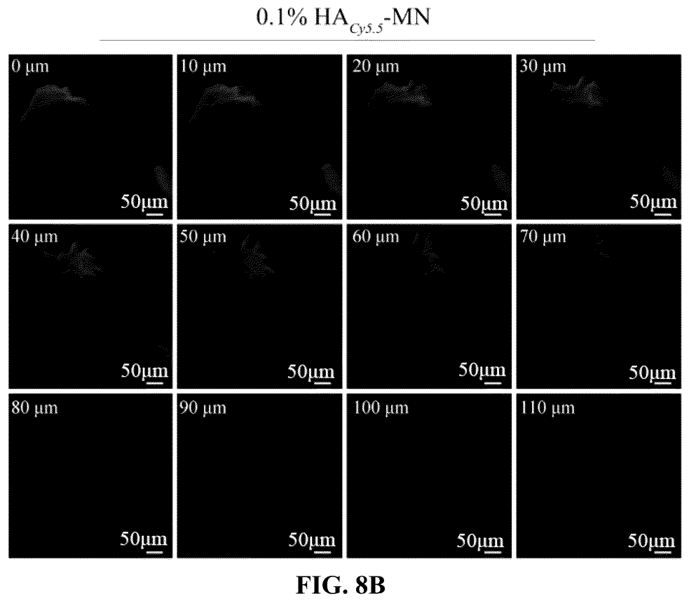

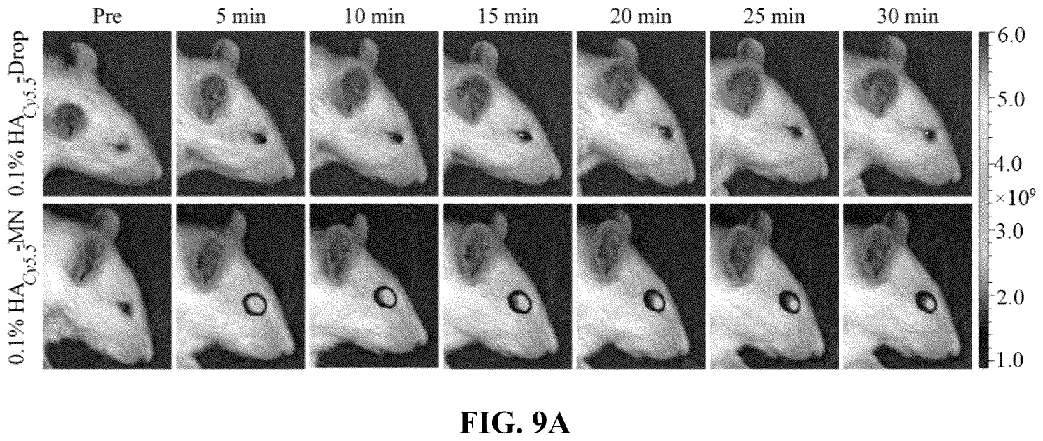

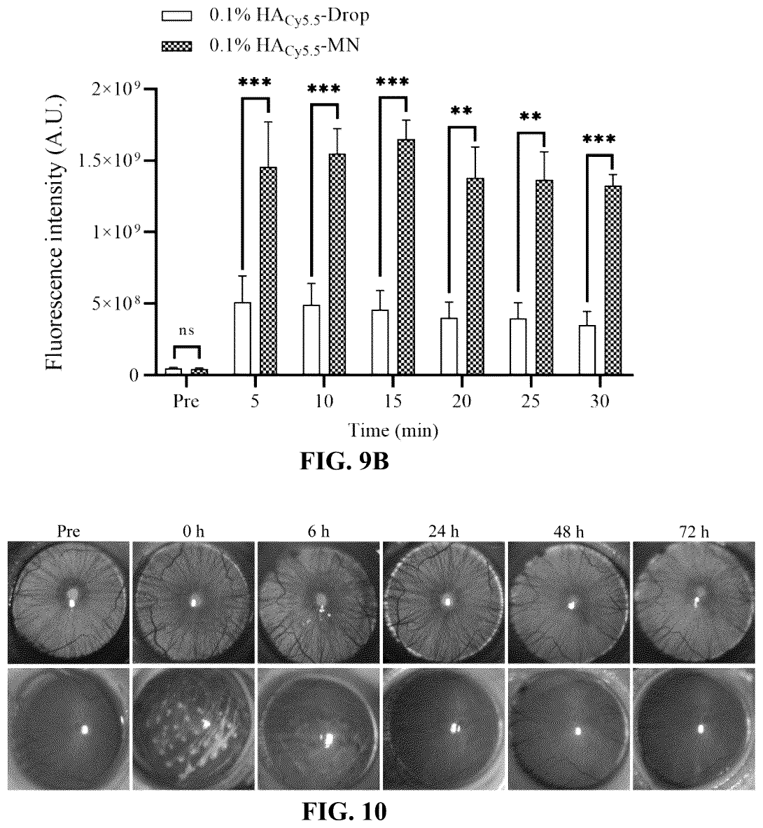

Grafting of a fluorescent molecule Cy5.5 on sodium hyaluronate to visibly track a delivery position and a delivery effect of sodium hyaluronate: 37.9 mg of sodium hyaluronate and 1.63 mg of Cy5.5 (a molar ratio of carboxyl in sodium hyaluronate to amino in Cy5.5 was 50:1) were weighed and added to 2 mL of ultrapure water, and stirring was conducted at room temperature for dissolution to obtain a sodium hyaluronate/Cy5.5 solution. 28.7 mg of 1-ethyl-3-(3-dimethylaminopropyl) carbodiimide hydrochloride (EDC) and 35 mg of N-hydroxysuccinimide (NHS) as catalysts were weighed and added to 1 mL of dimethyl sulfoxide, and uniform dissolution was conducted to obtain an EDC/NHS solution. The EDC/NHS solution was added to the sodium hyaluronate/Cy5.5 solution, and stirring and reaction was conducted at room temperature for 24 h to obtain a reaction solution. The reaction solution was further subjected to dialysis (molecular weight cut-off (MWCO)=3,500 Da) for 48 h and then lyophilized to obtain a sodium hyaluronate_{Cy5.5} powder. The sodium hyaluronate_{Cy5.5} powder was used to prepare an eye drop dosage form (0.1% HA_{Cy5.5}-Drop) and a microneedle patch dosage form (0.1% HA_{Cy5.5}-MN), respectively. a. The eye drop dosage form was applied to and the microneedle patch dosage form was allowed to penetrate through surfaces of eyeballs in rats, respectively. Then, the cornea was isolated, mounted, and photographed by z-axis scanning under a confocal microscope. As shown in FIGS. 8A to 8B: In the eye drop group, a weak fluorescence signal is detected in the shallow corneal layer. However, in the microneedle patch group, a fluorescence signal is significantly enhanced and penetrates into the deep corneal layer, indicating that the microneedle patch preparation can effectively improve the delivery efficiency and depth of sodium hyaluronate in corneal tissues. b. The anesthetized rats were administered with the eye drop through dropping and the microneedle patch through penetration, respectively. At 5 min, 10 min, 15 min, 20 min, 25 min, and 30 min, the rats were placed in an IVIS Spectrum imaging system, and imaged under the following parameters: a Cy5.5 optical filter (excitation wavelength: 675 nm/emission wavelength: 720 nm), an aperture value: 2, and a field of view: D. The LivingImage Software v.4.7.4 software was used to acquire images under the same camera settings. An ocular fluorescence intensity of a rat was measured with the Living Image software. As shown in FIGS. 9A to 9B: A weak fluorescence signal is detected in eyes of rats in the eye drop group, and the fluorescence signal is not evenly distributed in the eyes and is mostly concentrated in the interior corners of eyes. Moreover, the fluorescence signal is gradually weakened over time, and no fluorescence signal is found in eyes at 30 min. However, a fluorescence signal of the microneedle patch group covers the entire eye, and the fluorescence intensity is significantly enhanced. A high-intensity fluorescence signal still can be detected at 30 min. It indicates that the microneedle patch preparation can effectively improve the ocular delivery efficiency of sodium hyaluronate.

### 4. Ocular Safety of the Sodium Hyaluronate-Containing Microneedle Patch

The sodium hyaluronate-containing microneedle patch was attached to a surface of an eye of an anesthetized rat, gently pressed with the thumb for 60 s, and removed. The indicators such as ocular surface edema, inflammation, neovascularization, and corneal defect in rats were observed by a slit lamp. As shown in FIG. 10, the rat cornea is transparent and does not undergo edema, inflammation, and neovascularization. When the penetration of the microneedle patch is just finished, an array of punctate corneal defects occurs. Most of the defects have healed at 6 h, and the cornea is returned to an intact state at 24 h. It indicates that the microneedle patch has excellent animal ocular safety.

### 5. The Microneedle Patch Dosage Form can Effectively Improve the Corneal Repair Ability of Sodium Hyaluronate.

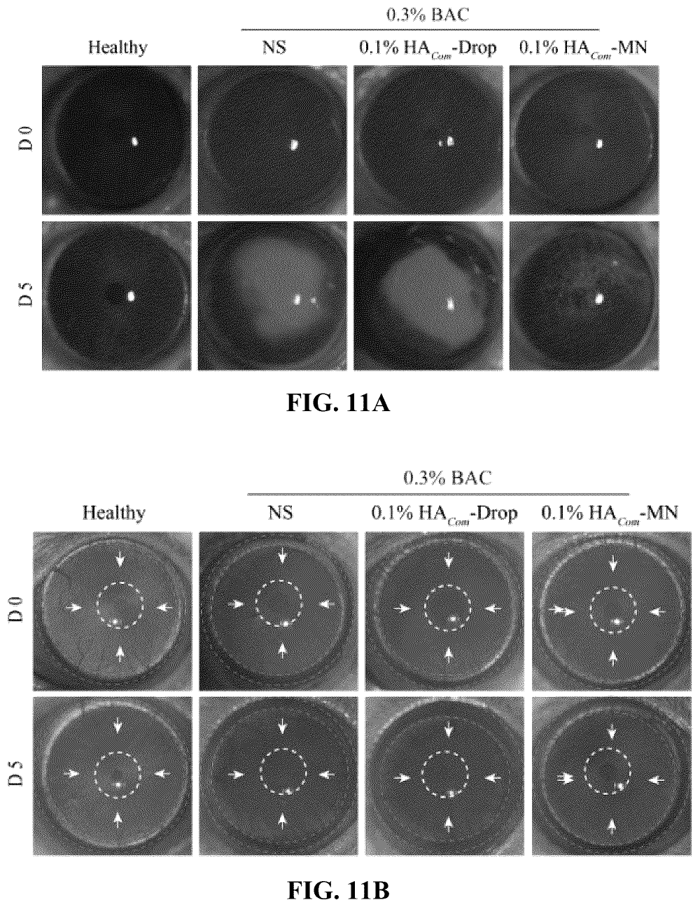

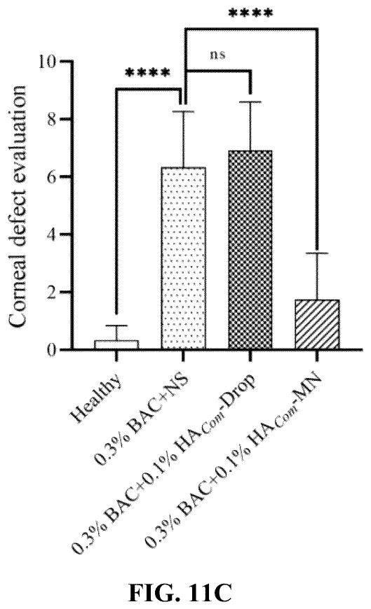

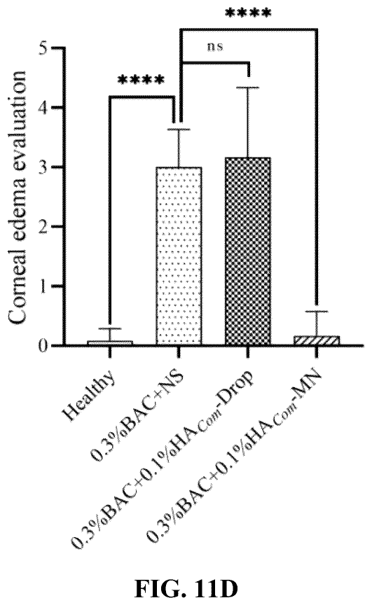

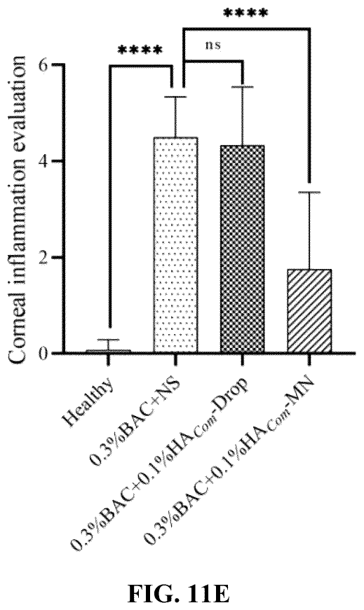

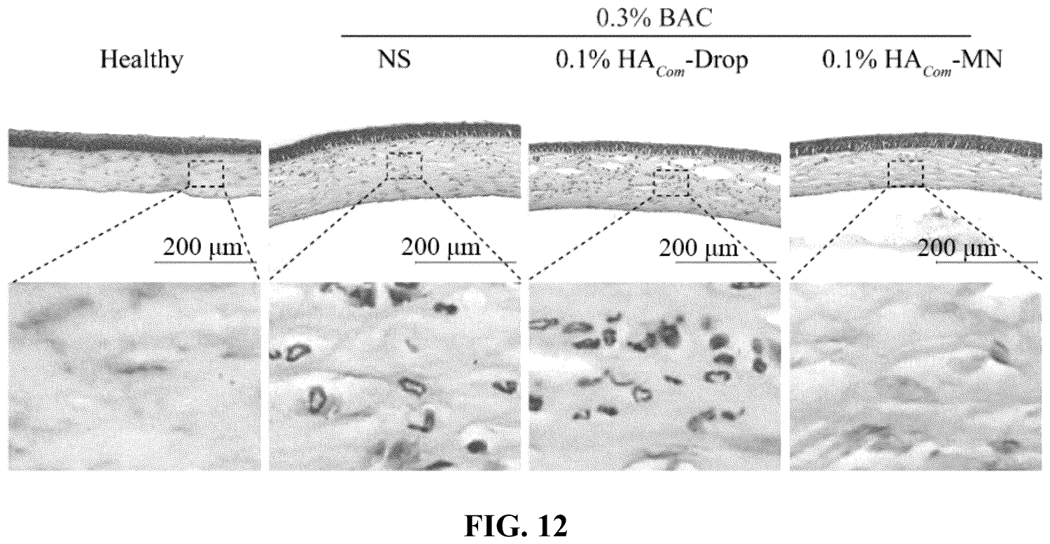

0.3% benzalkonium chloride (BAC) was applied to rat eyes once in both the morning and the evening consecutively for 5 days. A treatment manner was as follows: a. The sodium hyaluronate eye drop dosage form was dropped once in both the morning and the evening consecutively for 5 days, with 10 times in total. b. The sodium hyaluronate-containing microneedle patch dosage form was attached to a surface of an eye of a rat, gently pressed with the thumb for 60 s, and removed. The administration was conducted once only on the first day of modeling. The indicators such as ocular surface edema, inflammation, neovascularization, and corneal defect in rats were observed by a slit lamp. As shown in FIGS. 11A to 11E, the 0.3% BAC causes a large area of severe defects in the corneal epithelium of rats, and the corneal tissue undergoes symptoms such as pupil opacity, edema, inflammation, and neovascularization. The sodium hyaluronate eye drop cannot effectively improve the corneal wound degree. However, the sodium hyaluronate-containing microneedle patch can effectively repair the defective corneal epithelium, and the corneal tissue is transparent and does not have edema, inflammation, and neovascularization on day 5. It indicates that the microneedle patch can effectively treat a severe corneal wound. The rat cornea was further subjected to H&E staining to observe the inflammation. As shown in FIG. 12, the 0.3% BAC causes the infiltration of a large number of inflammatory cells in the corneal stroma of rats. The sodium hyaluronate eye drop cannot effectively reduce the infiltration of inflammatory cells in the corneal stroma. However, the sodium hyaluronate-containing microneedle patch can effectively reduce the infiltration of inflammatory cells in the corneal stroma. It further indicates that the microneedle patch dosage form can effectively exert the ability of sodium hyaluronate to repair the deep corneal layer.

In summary, the sodium hyaluronate-containing ophthalmic microneedle patch according to the present disclosure has the following advantages:

(1) The sodium hyaluronate-containing ophthalmic microneedle patch shows both strong mechanical properties and excellent flexibility, and exhibits prominent practicability for the treatment of uneven or curved tissues such as eye tissues.

(2) The sodium hyaluronate-containing ophthalmic microneedle patch only includes the components of sodium hyaluronate and PVA, which are simple and safe. The method for preparing the sodium hyaluronate-containing ophthalmic microneedle patch is simple, which is conducive to the large-scale mass production.

(3) A treatment process of the sodium hyaluronate-containing ophthalmic microneedle patch is simple. The microneedle patch only needs to be attached to a corneal surface and gently pressed with the thumb for 60 s, and then can be discarded after single use, which is enough to make the corneal tissue fully absorb sodium hyaluronate. Thus, the sodium hyaluronate-containing ophthalmic microneedle patch effectively overcomes the dilemma that the too-frequent dropping of eye drops would result in poor patient compliance.

(4) The microneedle patch has prominent biosafety. The minimally-invasive and painless characteristics of the microneedle patch can effectively reduce the psychological disorders of patients caused by eye injection, for example.

(5) The microneedle patch shows excellent mechanical properties, and can effectively break the physical barrier of a corneal epithelial layer.

(6) The microneedle patch can efficiently deliver sodium hyaluronate into the corneal epithelial layer and stroma, and improve the bioavailability of sodium hyaluronate eye drops at an ocular tissue.

(7) The microneedle patch can effectively repair the refractory severe corneal wound, including: repairing the severe corneal epithelial defect, reducing the corneal tissue edema and neovascularization, improving the clarity of a pupil in a center of a cornea, and inhibiting the infiltration of inflammatory cells in a corneal stroma.

The above are only preferred embodiments of the present disclosure, and are not intended to limit the claimed scope of the present disclosure. Therefore, equivalent changes made according to the claims of the present disclosure are still within the scope of the present disclosure.

What is claimed is:

1. A method for preparing a sodium hyaluronate-containing ophthalmic microneedle patch, comprising:
S1, preparing a microneedle patch matrix solution, wherein the microneedle patch matrix solution comprises sodium hyaluronate and polyvinyl alcohol (PVA); and
S2, dropping the microneedle patch matrix solution into a microneedle patch mold, drying, and then demolding to obtain the sodium hyaluronate-containing ophthalmic microneedle patch.

2. The method for preparing the sodium hyaluronate-containing ophthalmic microneedle patch of claim 1, wherein a total matrix solid content in the microneedle patch matrix solution is in a range of 5% to 15%.

3. The method for preparing the sodium hyaluronate-containing ophthalmic microneedle patch of claim 2, wherein the total matrix solid content in the microneedle patch matrix solution is 10%.

4. The method for preparing the sodium hyaluronate-containing ophthalmic microneedle patch of claim 2, wherein the PVA in the microneedle patch matrix solution accounts for 40% to 99% of the total matrix solid content.

5. A sodium hyaluronate-containing ophthalmic microneedle patch prepared by the method for preparing the sodium hyaluronate-containing ophthalmic microneedle patch of claim 1.

6. A method for treating a severe corneal wound, comprising: administering the sodium hyaluronate-containing ophthalmic microneedle patch of claim 5 in a therapeutic dose to subjects in need thereof.

7. The method of claim 6, wherein the severe corneal wound comprises at least one selected from the group consisting of the severe corneal wound caused by long-term preservative application, severe dry eye disease, the severe corneal wound caused by alkali burn, the severe corneal wound caused by a mechanical trauma, bacterial keratitis, and fungal keratitis.

8. The sodium hyaluronate-containing ophthalmic microneedle patch of claim 5, wherein a total matrix solid content in the microneedle patch matrix solution is in a range of 5% to 15%.

9. The sodium hyaluronate-containing ophthalmic microneedle patch of claim 8, wherein the total matrix solid content in the microneedle patch matrix solution is 10%.

10. The sodium hyaluronate-containing ophthalmic microneedle patch of claim 8, wherein the PVA in the microneedle patch matrix solution accounts for 40% to 99% of the total matrix solid content.
